(19) United States
(12) Patent Application Publication
(10) Pub. No.: US 2023/0346247 A1
(43) Pub. Date: Nov. 2, 2023

(54) SYSTEM AND METHOD FOR MAGNETIC RESONANCE IMAGING USING SHAPED RADIO FREQUENCY PULSES

(71) Applicant: The General Hospital Corporation, Boston, MA (US)

(72) Inventors: Bastien Guerin, Somerville, MA (US); Lawrence L. Wald, Cambridge, MA (US); Jason Stockmann, Cambridge, MA (US)

(21) Appl. No.: 18/311,145

(22) Filed: May 2, 2023

Related U.S. Application Data
(60) Provisional application No. 63/337,281, filed on May 2, 2022.

(51) Int. Cl. A61B 5/055; G01R 33/561

(52) U.S. Cl. CPC A61B 5/055; G01R 33/5612

(57) ABSTRACT

A system and method are described for MRI excitation pulse design. The system can include a magnetic system that produces a main magnetic field over a portion of a subject for MRI imaging. The system can also include an RF system configured to transmit and receive an RF or B₁⁺ field across at least a target region within the subject. The system may further include a gradient system configured to spatially encode the B₁⁺ field using a gradient waveform. The system may also include a control system, which can be configured to control the RF system in order to generate an RF excitation pulse. The excitation pulse includes freely-shaped RF waveforms, gradient waveforms and, potentially shim array waveforms, selected by penalizing deviation of a flip-angle from a target distribution in order to achieve a target magnetization profile. The method can be applied to 3D and 2D slice-selective excitation and refocusing.

### CROSS-REFERENCE TO RELATED APPLICATIONS

This application is based on and claims priority to U.S. Provisional Patent Application No. 63/337,281 filed on May 2, 2022, the entire contents of which is incorporated herein by reference.

### STATEMENT REGARDING FEDERALLY SPONSORED RESEARCH

This invention was made with government support under R01EB006847 and R00EB019482 awarded by the National Institutes of Health. The government has certain rights in the invention.

### BACKGROUND

Although ultra-high field magnetic resonance imaging (MRI) produces high signal to noise ratio (SNR), it is also associated with inhomogeneous flip angles of spins across the volume or slice of interest. This is a well-known problem usually referred to as the “B₁⁺ problem”, and is due to the fact that, as the field strength increases, the wavelength of the radiofrequency (RF) radiation becomes comparable to the size of the body part imaged.

For example, the in-tissue wavelength of the RF radiation at 3 Tesla is ˜40 cm. Therefore, when imaging the torso at 3 Tesla, wavelength effects are visible on MR images. At 7 Tesla, the in-tissue wavelength of the MRI RF radiation is ˜16 cm. Therefore, at 7 Tesla wavelength effects start to be visible when imaging the head, and the effect is even more pronounced when imaging the torso at 7 Tesla.

Because the RF field produced by the MRI transmit coil, which is called the B₁⁺ field, is responsible for tipping the spins in the transverse plane so that we can generate a signal, this phenomenon causes non-uniform contrast weighting across the field-of-view. This is a major confounding problem that negatively impacts the contrast of MR images, as this source of contrast variation has nothing to do with the MRI properties of the patient. This problem is especially pronounced at high magnetic fields, such as 3 Tesla and 7 Tesla, and above. As increasing field strengths find broad clinical utilization, the market for those machines expands and it becomes more and more critical to find simple, cost-effective, easily deployable solutions to the “B₁⁺ problem”.

### SUMMARY OF THE DISCLOSURE

The present disclosure addresses the aforementioned drawbacks by providing a system and method for controlling against the B₁⁺ problem, even at higher field strengths. For example, using the systems and methods provided herein excitation pulses can be designed that are not initially constrained to a particular shape. These freely-shaped RF pulses can be designed for 3D and slice-selective 2D MRI to control against the B₁⁺ problem, even at higher field strengths. Thus, excitation pulses can be designed that control against the B₁⁺ problem even when using a single radiofrequency coil and starting from arbitrarily-shaped RF pulse waveforms.

In one aspect of the present disclosure, a method for performing an MRI process is presented. The method can include determining a target excitation region for imaging a subject located within the MRI system. The method can also include determining a target magnetization profile for the imaging process. The method can also include accessing field map data for the MRI system with a computer system, which may include a B₀ field map and a B₁⁺ field map measured for an RF transmit coil. The computer system can be used to design an RF pulse waveform that has a shape selected using an objective function. The objective function can include at least one constraint and can be configured to control a deviation from the target magnetization profile within the target excitation region or an RF power requirement level or both. Such optimization can be based on the field map data. The designed RF pulse waveform can be communicated to the MRI system in order to perform imaging of the subject.

In another aspect of the present disclosure, an MRI system is provided. The system can include a magnetic system that can produce a main magnetic field over at least a portion of a subject for MRI imaging. The system can also include an RF system that is configured to transmit and receive an RF or B₁⁺ field across at least a target region within the portion of the subject. The system may further include a gradient system that is configured to spatially encode the B₁⁺ field using a gradient waveform. The system may also include a control system. The control system can be configured to control the RF system in order to generate an RF excitation pulse. The RF excitation pulse can have a shape that is selected by penalizing deviation of a flip-angle of the RF excitation pulse from a target flip-angle distribution in order to achieve a target magnetization profile in the target region.

### BRIEF DESCRIPTION OF THE DRAWINGS

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

FIG. 1 is a block diagram that illustrates a process that produces desired excitation pulses in accordance with the present disclosure.

FIG. 2 is a flow chart that illustrates a method for acquiring magnetic resonance imaging data using excitation pulses in accordance with the present disclosure.

FIG. 3 shows an example of flip angle profiles used to compare a conventional approach and pulse design approach in accordance with some aspects of the present disclosure.

FIG. 4 shows an example of slice profiles used to compare a conventional approach, 2-spoke sinc approach, and pulse design approach in accordance with some aspects of the present disclosure.

FIG. 5 shows an example of flip angle profiles used to compare a conventional approach and pulse design approach applied to new subjects in accordance with some aspects of the present disclosure.

FIG. 6 shows an example of the tradeoff between specific absorption rate and flip angle homogeneity used to compare a conventional approach, parallel transmit (pTx) approach, and pulse design approach in accordance with some aspects of the present disclosure.

FIG. 7 is a block diagram of an example magnetic resonance imaging (“MRI”) system that can implement the methods described in the present disclosure.

FIG. 8 is a block diagram of an example MRI excitation pulse design system that can implement the methods of the present disclosure.

FIG. 9 is a block diagram of example components that can implement the system of FIG. 8.

### DETAILED DESCRIPTION

Before any aspects of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Also as used herein, unless otherwise limited or defined, “or” and “and/or” indicate a non-exclusive list of components or operations that can be present in any variety of combinations, rather than an exclusive list of components that can be present only as alternatives to each other. For example, a list of “A, B, or C” or “A, B, and/or C” indicates options of: A; B; C; A and B; A and C; B and C; and A, B, and C. Correspondingly, the term “or” as used herein is intended to indicate exclusive alternatives only when preceded by terms of exclusivity, such as “either,” “one of,” “only one of,” or “exactly one of.” For example, a list of “one of A, B, or C” indicates options of: A, but not B and C; B, but not A and C; and C, but not A and B. A list preceded by “one or more” (and variations thereon) and including “or” to separate listed elements indicates options of one or more of any or all of the listed elements. For example, the phrases “one or more of A, B, or C” and “at least one of A, B, or C” indicate options of: one or more A; one or more B; one or more C; one or more A and one or more B; one or more B and one or more C; one or more A and one or more C; and one or more of A, one or more of B, and one or more of C. Similarly, a list preceded by “a plurality of” (and variations thereon) and including “or” to separate listed elements indicates options of multiple instances of any or all of the listed elements. For example, the phrases “a plurality of A, B, or C” and “two or more of A, B, or C” indicate options of: A and B; B and C; A and C; and A, B, and C.

In MRI, the flip-angle of spins across the volume or slice or interest is generally not uniform, which is exacerbated at increasing field strengths. The most well-known solution to this problem is parallel transmission (pTx). In pTx, several transmit channels are used simultaneously to excite the MRI signal. Each channel has a highly nonuniform profile, but RF pulses can be designed that combine these profiles in such a way that the flip angle is uniform at the end of the excitation pulse.

For 3D imaging, whereby the entire object or a substantial part of the object is excited at once (as opposed to exciting only a slice), a well-known pTx pulse is the kT-point pulse. In the kT-point pulse, transmit energy is deposited at discrete locations in transmit k-space and the amplitude and phases of these discrete RF bursts are optimized by a computer program to yield a uniform flip angle distribution. The equivalent of kT-points for slice-excitation is the so-called “spokes pulse”, whereby a sinc-shaped sub-pulse is played at different transmit k-space locations. Like the kT-point bursts, optimization of the amplitude and phase of the different spokes allows for the creation of a uniform flip angle distribution while exciting a slice thanks to the imposed sinc shape of the spoke sub-pulses. A common feature of the kT-point and spoke pulses is that they use fixed sub-pulse shapes. For example, rectangular sub-pulses are typically used for kT-points, and sinc pulse are typically used for spokes.

Unfortunately, the use of pTx requires specialized pTx systems, which are uncommon, especially in clinical settings, complex, and expensive. Moreover, conventional pTx methods provide limited flexibility as conventional pulses allow for control of either the slice profile or the in-slice flip-angle distribution but not at the same time. Thus, the present disclosure describes a system and method that provide improved excitation homogeneity using widely available conventional RF systems or may outperform conventional pTx approaches.

A system and method are described herein for MRI excitation pulse design that generates an RF pulse waveform, which generates a field, and a gradient waveform, which provides spatial localization of the field. The described system and method provide a new class of freely-shaped pulses that provide increased degrees of freedom and outperform the kT-point and spoke pulses for slice-selective and 3D excitation, respectively. These freely-shaped pulses are designed to generate a uniform magnetization or flip angle distribution or reduce power requirements, or both. The freely-shaped pulses can be designed for 3D excitation or for slice excitations. Advantageously, these freely-shaped pulses can be played by a standard MRI system equipped with a single channel transmit coil or on an advanced MRI system that is equipped with a pTx system.

These freely-shaped pulses are referred to as such because they are designed with more excitation degrees of freedoms than traditional pulses, such as kT-point pulses. The disclosed methods can take advantage of degrees of freedom provided by freely shaped RF pulses, a gradient system, and an optional shim system, which can achieve target excitation within desired operational constraints, even without the use of pTx. Therefore, these carefully-designed, freely-shaped excitation pulses can be readily played on standard MRI hardware that is equipped with a single RF transmit coil. In particular, the freely-shaped pulses can be performed by a bird cage coil, which is ubiquitous for most standard MRI scanners. In contrast, only a small number of MRI scanners have pTx systems, which has prohibited the clinical adoption of pTx methods. As a result, the freely-shaped pulses can be more widely used than pTx pulses because they can easily be distributed to different sites and scanners. Moreover, the freely-shaped pulses can also be played on a pTx systems, when available, to further improve the pulse design. Using multiple RF transmit coils in pTx mode, the freely-shaped excitation pulses outperform conventional kT-point and spoke pulses.

Optionally, the system and method can further leverage the degrees of freedom provided by shim systems, when available. The freely-shaped pulses can be designed in conjunction with shim waveforms that can improve the quality of flip-angle excitations and refocusing and/or reduce power consumption. However, if a shim system is unavailable, the freely-shaped excitation pulses can still leverage the degrees of freedom provided by the freely shaped RF pulses and linear gradient fields available on standard MRI systems.

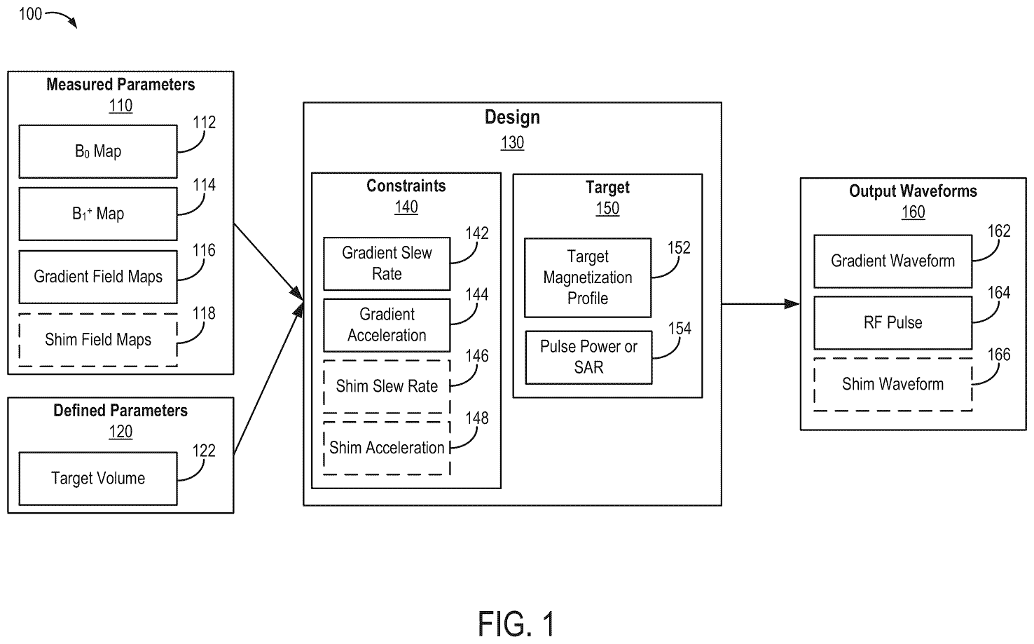

The process 100 illustrated in FIG. 1 can be performed on a computer system to design freely-shaped excitation pulses. The freely-shaped pulses may advantageously be used at high field strengths, such as 3 T, 7 T, or others. to mitigate effects of inhomogeneity. However, the system and methods described herein may also be applied at low field strengths, such as 1.5 T, and the like. Along with RF pulse waveforms, the design process may output gradient waveforms to achieve a target magnetization profile. These waveforms can be stored for use with an MRI system.

In the design process 100, measured parameters 110 and defined parameters 120 can act as inputs to a design 130 that overcomes the drawbacks described above, including the B₁⁺ problem. The design 130 may be referred to as an “optimization” and may yield an optimized RF pulse or pulses. However, the present disclosure also recognizes that freely-shaped RF pulses that are not or not quite “optimal” in a mathematical sense may also be useful. Furthermore, “optimization” can yield different results depending upon the specific constraints used or the weights applied to generate the output. Thus, while the systems and methods provided herein can be used to generate optimal RF pulses, such pulses will be more generally described as freely-shaped RF pulses, recognizing that optimization or a particular form of optimization may or may not be desired in any particular execution of the systems and methods of the present disclosure.

In one non-limiting example, the design 130 can design a gradient waveform 162 and/or an RF pulse waveform 164 as output waveforms 160. The design 130 can also optionally include the design of a shim waveform 166. The design 130 can be formed as an objective function that includes one or more constraints 140 and a desired target 150. For example, the objective function may penalize deviation of the achieved flip angle from the ideal target flip angle distribution (either across a 3D target volume or 2D slice profile) or the power requirements, or both. The generated pulses do not need to be parameterized and can take, for example, any of a wide variety of shapes that benefit reduction or minimization of the cost function. In particular, the generated pulses do not need to be constrained to predefined pulses, such as sinc pulses, rectangular pulses, Shinnar-Le Roux pulses, or the like, which are typically used for pTx. The absence of assumption on the specific shape of these pulses is one reason why the pulses of the present disclosure outperform conventional strategies, as this “frees up” excitation degrees of freedom that are otherwise not accessible because of the choice of a specific pulse (or sub-pulse) shape (e.g., square pulse, sinc pulse, or the like).

The design 130 can use measured parameters 110 and defined parameters 120 as inputs. For example, the defined parameters may include a user-defined target volume 122 or target imaging region. The target volume 122 may be a target 2D slice profile for 2D MRI, 3D volume for 3D MRI, or another arbitrarily shaped imaging volume or region. For example, the target volume 122 may be defined as a 5 mm rectangular slice. It may also be defined in order to selectively excite only the brain region of a subject. The target volume 122 may be a slice in x, y, z, or another oblique axis, a prism shape, spherical shape, or other shape. The target volume 122 may also be multiple 2D slice profiles or 3D slabs separated in space, for example, to facilitate simultaneous multi-slice imaging.

The measured parameters 110 may include measured field map data, including a B₀ map 112 and a B₁⁺ map 114 measured for the corresponding MRI system. The measured parameters 110 may further include gradient field maps 116 that characterize the response of a gradient system. The measured parameters may optionally also include shim field maps 118 that characterize the B₀ field while employing a shim system, when available.

A B₀ map 112 is a map of the frequency deviation from the ideal Larmor frequency (e.g., ˜128 MHz at 3 Tesla, ˜300 MHz at 7 Tesla, and so on) and is dependent on the MRI system (e.g., superconducting loop geometry and coil performance, permanent magnet geometry, system temperature, placement of passive shims, active shim waveforms of the MRI system, and the like), the load of the MRI system (e.g., the subject, implants, other hardware, and the like), and otherwise-generated B₀ fields (e.g. eddy currents and the like). The B₀ maps 112 can be measured by standard receive coils, such as surface coils or a body coil, or other employed receive RF coils. The B₀ maps 112 can be measured by standard B₀ mapping methods, such as using a double-echo gradient echo sequence, or other methods.

A B₁⁺ map 114 indicates the RF field achieved for a given patient using a particular RF transmit coil. The B₁⁺ maps 114 may be measured using B₁-mapping sequences and generated with standard B₁ mapping methods or by other methods known in the art.

Gradient field maps 116 describe a spatial B₀ field achieved while a gradient system is in use. For example, a gradient field map 116 can be generated by measuring B₀ in a region of interest while a known current is applied to each gradient coil of the gradient system. For example, the gradient system can be used to provide a prescribed linear gradient along the x direction with a known slope, such as 20 mT/m. B₀ mapping can be used to measure the achieved gradient with respect to the prescribed gradient. Standard B₀ mapping may be used to measure the gradient field map.

If a shim system is available, the shim field map can be measured to characterize the B₀ field achieved when a shim system is used to adjust the B₀ field. For example, the B₀ field can be measured for each coil while each coil of the shim array is provided with a current, such as 1 A or another current. The shim field map 118 can also be measured by supplying other combinations of coils with varying known currents. In this way, the shim system can be calibrated to account for the geometry and performance of the shim system.

While the gradient field map 116 and shim field map 118 are system specific, the gradient system and shim system calibrations may be considered independently of the patient or MRI system load. Thus, the gradient field maps 116 and shim field maps 118 may advantageously be measured a single time for each system and used repeatedly in the design process to inform the gradient and shim waveforms, as will be described in further detail below.

In contrast, the B₀ map 112 and B₁⁺ map 114 depend not only on the MRI system, but also on the shape and body composition of the particular subject being imaged. Therefore, the B₀ map 112 and B₁⁺ map 114 may be uniquely measured for a particular subject within the MRI system such that the design can be fine-tuned for the given subject. Alternatively, the B₀ map 112 or the B₁⁺ map 114 may include B₀ and B₁⁺ maps measured by the MRI system for a variety of different subjects. These B₀ and B₁⁺ maps can be concatenated to form the B₀ map 112 or B₁⁺ map 114, respectively, used in the design 130. For example, an M-patients database can be used in which the input to the design can be M B₁⁺ maps and M B₀ maps. In this way, the pulses can be generated prior to the scan session for flexibility and ease of use. Using a concatenated B₀ map 112 and B₁⁺ map 114 can provide pulses that are robust to normal variations of B₁⁺ and B₀ across the patient population. These pulses may be readily applicable to new patients without the need for acquisition of new B₀ and B₁⁺ maps or additional computation. This strategy can provide universal pulses that can conveniently be used for new subjects and easily be distributed to different sites.

The design 130 may be performed for a single channel RF transmit coil or a pTx RF system with two or more channels. If a single coil is being used, the B₁⁺ map will include one B₁⁺ map per subject. Otherwise, if pTx is being used, the B₁⁺ map 114 should include a B₁⁺ map for each channel of the RF coil array (i.e., N B₁⁺ maps 114 in an N-channel pTx system).

While there is no specific shape imposed on the pulses, it can be helpful to add constraints 140 to the design method. One such constraint is the slew-rate 142 (or rate-of-change) of the gradient waveforms. This quantity is limited by the inductance of the gradient system and the total voltage available from the gradient power amplifier and cannot exceed a certain value for a given hardware system. Therefore, it may be constrained in the design procedure so that the pulse can be used on the scanner. Another useful constraint may be the acceleration 144 (the rate of change of the rate of change) of the gradient waveform. Limiting the gradient waveform acceleration 144 yields smoother waveforms that can be reliably performed by the gradient system. Other constraints 140 can be included based on system limitations, safety concerns, or other desired characteristics of the excitation waveforms. For example, constraints may include peak power, average power, local specific absorption rate (SAR), global SAR, or the like. Other design constraints 140 include shim slew rate 146 and shim acceleration 148 which will be described further below.

The design 130 can include an objective function that is reduced or minimized based on a predefined target 150. For example, the target 150 may be to minimize or control the deviation from a target magnetization profile 152, to minimize a pulse power or SAR 154 required to generate the excitation pulses, or a combination of both. For example, the target magnetization profile 152 may be defined based on a flip angle, a Be magnetization profile, or a resultant magnetization profile (e.g., M_{xy} and M_{z}), as these parameters are inter-related. As a non-limiting example, the target magnetization profile 152 may be defined as a homogeneous flip angle, such as 10°, 20°, 30°, 40°, 50°, 60°, 70°, 80°, 90°, 100°, 110°, 120°, 130°, 140°, 150°, 160°, 170°, 180°, or any other desired flip angle within the target volume 122. The target magnetization profile 152 may also be defined as a non-homogeneous flip angle profile, such as linearly varying flip angle or another desired flip angle profile.

The target magnetization profile 152 may also be defined based on a desired B₁⁺ field. For example, the target magnetization profile 152 may be defined as a homogeneous or other B₁⁺ profile over the target volume 122. The target B₁⁺ profile may include a magnitude and a phase component. Similarly, a resultant magnetization (M) can act as the target magnetization profile 152. The resultant magnetization may include a magnitude and phase component. The magnetization may also be described based on transverse magnetization (M_{xy}), which is dependent of phase, or longitudinal magnetization (M_{z}), which is independent of phase. For example, M_{z} may be used when the phase is not of interest, and M_{xy} may be used when phase is of interest, for example in turbo spin-echo sequences where the Carr-Purcell-Meiboom-Gill condition imposes a fixed relationship between the phases of the excitation and the refocusing pulses.

The target magnetization profile 152 may also define a desired magnetization, B₁⁺, or flip angle in a region outside of the target volume 122. For example, the target magnetization profile 152 can constrain the whole region outside of the target volume 122 to have a flip angle of 0° to avoid excitation outside of the volume or slice of interest.

The pulse power or SAR 154 may include a global measure of pulse power or SAR, such as maximum/peak or average, or a local measure of pulse power or SAR, such as a SAR map. Additionally or alternatively, a pulse power or SAR can be used as a constraint 140, such as by constraining the maximum pulse power or SAR over the subject.

The design process 100 will provide 3D or 2D slice excitations that outperform standard methods. The output waveforms 160 can generate significantly better flip angle distributions, i.e., closer to the ideal distribution, while reducing the power requirement. As a result, the user is free to trade the additional degrees of freedom unlocked by the non-parametric pulse design for 1) improved flip angle performance while maintaining pulse power (compared to the conventional excitation), 2) decreased power consumption while maintaining the same flip angle performance, or 3) improving the flip angle performance while decreasing the power consumption.

Power is related to the specific absorption rate (SAR), which is regulated by the Federal Drug Administration to guarantee patient safety. Therefore, reducing the pulse power may allow 1) increasing the data acquisition rate (i.e., number of excitations per second) while maintaining the SAR level, 2) decreasing the SAR level, thus improving patient safety, while maintaining the data acquisition rate, or 3) a combination thereof. In a single channel case, the pulse power is proportional to SAR, and thus, minimizing the power is equivalent to minimizing SAR. Moreover, power control is simpler using a single channel RF transmit system compared to using multiple RF channels (pTx). Therefore, in single channel use, the method can provide a significant safety advantage and easier use. Alternatively, in parallel transmit mode, the design 130 can minimize or control power or directly minimize the SAR as an alternative to pulse power.

As discussed above, the target volume 122 may be a slice profile to facilitate 2D MRI or a 3D imaging volume to facilitate 3D MRI. For 3D imaging, the objective function of the design 130 can penalize deviations of the flip angle over the whole target volume 122. For example, the target volume 122 may be defined as the whole head of the subject or a subset region that is of specific interest, such as the brain. In this case, the objective function may only consider the target magnetization 152 within the target volume 122 and may be ignorant to the target magnetization 152 (e.g., B₁⁺, magnetization, or flip angle) outside of the target volume 122.

In contrast, it may be beneficial to include a desired target magnetization 152, such as flip angle profile, for the region outside the target volume (i.e., flip angle=0). In one non-limiting example, the target volume may be defined as a slice and the objective function may include at least two terms. The first term can be used to represent the in-slice term, which penalizes deviations of the flip angle from the desired flip angle in the specified slice profile. The second term may represent the region outside of the excited slice and penalize any flip angle greater than 0. In practice, even small flip angle deviations from the ideal zero value can create large transverse magnetization (i.e., in the X-Y plane), which can corrupt the desired signal. Thus, it may be useful to penalize this transverse magnetization outside the target volume 122 in the design 130 problem to effectively suppress the transverse magnetization. Similarly in the 3D case, the design 130 can include a second term that represents a region outside of the target volume 122, which may be beneficial for acquisition by reducing the field of view (FOV), and the like.

As a non-limiting example, the design 130 may be formed as a magnitude least squares of the transverse magnetization (M_{xy}). The phase can also be included in the objective function. For example, the target magnetization profile 152 may be defined as a constant magnitude and phase in space. For example, the phase target may be defined as that produced by a birdcage coil.

Inside the slice or volume of interest, the target magnetization profile 152 of the objective function may include a longitudinal magnetization (i.e. along the Z-axis) term as a surrogate for flip angle based on their trigonometric relationship. Longitudinal magnetization is independent of the phase of the magnetization, so this strategy is equivalent to performing a magnitude least-squares optimization, which is known to have superior performance compared to a least-squares optimization. However, the objective function may alternatively use flip angle directly or may use phase-dependent transverse magnetization.

In some MRI pulse sequences, the magnetization phase is of interest. For example, in turbo spin-echo sequences, the Carr-Purcell-Meiboom-Gill condition imposes a fixed relationship between the phase of the 90° excitation and the 180° refocusing pulses. In these cases, the objective function may be based on the transverse magnetization and include the phase for the in-slice or in-volume terms.

Advantageously, the design process 100 allows for full control of the slice profile and the flip angle distribution. In contrast, conventional approaches typically allow for independent control of the slice profile or the flip angle distribution in the slice but not both at the same time. For example, the Shinnar-Le Roux class of pulses offer excellent slice profiles but cannot correct the in-slice flip angle inhomogeneity at 3 Tesla or 7 Tesla. On the other hand, spoke pulses can generate a uniform flip angle distribution in-slice but do not control the slice profile and thus, often yield poor slice profiles.

In addition to the traditional degrees of freedom that are used to excite the spins in MRI, namely the parameters of the RF coil, RF waveform, and 3-axis gradient system, the design process 100 can leverage non-traditional degrees of freedom to further improve the design. For example, the design 130 may include degrees of freedom provided by a shim system, or active shim system, when available. Shim systems are typically used to obtain a more uniform B₀ field in a region of interest, which may include improving upon the passive B₀-shimming. The shim system may be external to and/or integrated with the MRI system. In general, a shim system includes one or more shim coils that can surround the whole body or anatomy of interest or be placed near the body similar to RF surface coils. The shim system may include multiple shim coils arranged in an array. Several different shim systems can be employed. However, it may be advantageous to use a shim system that allows for high update rate to achieve shim waveforms that are minimally constrained. As a non-limiting example, the shim system may advantageously update with a 50-100 kHz bandwidth.

As one non-limiting example, the shim system may include a spherical harmonic (SH) system capable of providing shims with high-order basis functions (e.g., up to 5-th order). Such systems may be available on standard MRI systems. The spherical harmonic shim systems may be built into the gradient coils of the MRI system or may include shim insert coils. In use, these systems may provide static shimming that is constant throughout an MRI exam. The SH system may also provide dynamic shimming in which the shim currents can be dynamically switched in order to improve shimming for each individual slice or slab.

Additionally or alternatively, the shim system may include advanced shim systems. For example, the advanced shim system may include single or multi-coil shim arrays. The multi-coil shim arrays may include small loops associated with low inductances. As a non-limiting example, these multi-coil shim arrays may include AC/DC coils. The multi-coil shim arrays may achieve lower inductance and provide faster update rates than standard SH shim systems. The loop coils may have circular or nearly circular shapes or may have other irregular shapes. These shim arrays can be patterned around the anatomical region of interest to generate B₀ shim field at high spatial orders. The coils may be positioned near the body, similar to RF receive surface coils, or may be placed around the body, similar to volume coils. The coils may be energized by DC or low-frequency (e.g., <1 MHz) current waveforms provided by low-voltage amplifiers and can be switched rapidly to achieve dynamic shimming. These shim arrays may share the structure, or loop, of receive RF coil elements. As another example, the advanced shim system can include dedicated multi-turn (e.g., >20 turns) shim coils that can be placed outside of the transmit coil shield, for example. As a non-limiting example, such an array may include 48 channels, each with 100 loop turns, arranged in a cylindrical shim array surrounding the body or anatomy of interest.

Use of the designed output waveforms 160 does not require the use of a shim array. However, the performance of the design process 100 may be improved when using this additional piece of hardware. In particular, the design 130 can generate a shim waveform 166 along with the generated gradient waveform 162 and RF pulse waveform 164 in order to improve the quality of the flip angle distribution. The use of the shim system may be particularly valuable in the case of 2D imaging with slice selection. When the shim waveform 166 is included in the design 130 problem, additional constraints 140 on the shim waveform 166 can be included to ensure practical results. For example, these constraints 140 may include the slew rate 146 of the shim current waveform 166 or the acceleration 148 of the shim current waveform 166. Such constraints can ensure that the energy of the shim current waveforms 166 is maintained within the bandwidth of the shim array amplifier.

Advantageously, utilizing the degrees of freedom provided by the gradient system and shim system does not affect the SAR of the RF excitation. In contrast, pTx methods achieve additional degrees of freedom by using additional RF transmit channels that can greatly impact SAR. Such increase in SAR can increase design and system complexity and may cause safety concerns.

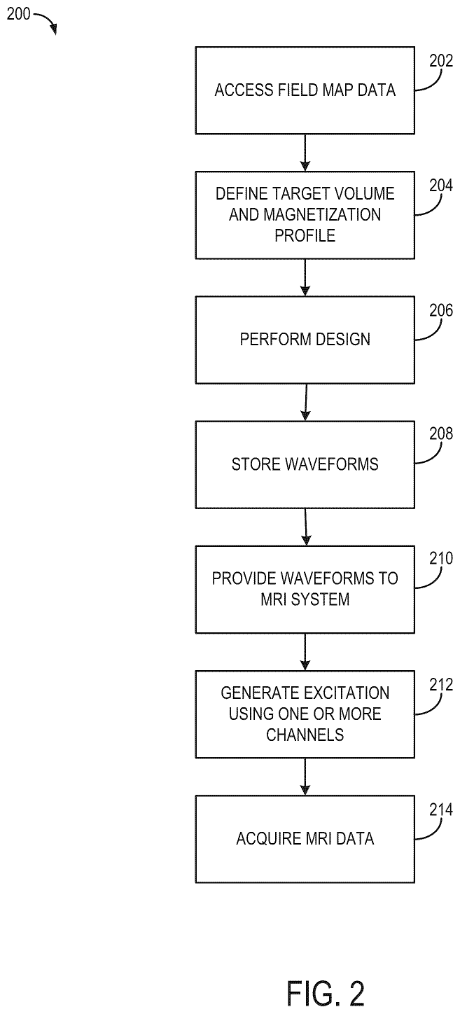

Referring now to FIG. 2, the process 200 can be used to generate and apply freely-shaped excitation pulses with increased degrees of freedom for MRI. The process may begin in block 202 by accessing field map data, such as B₀ maps, B₁⁺ maps, shim field maps, or gradient field maps. For example, accessing field map data may include measuring B₀ and B₁⁺ maps using the MRI system for a particular subject. The B₀ and B₁⁺ maps may also be stored in the memory of a computer system and accessed by a computer system. The B₀ and B₁⁺ maps may include maps measured for the particular subject or may be a group of maps measured for a wide range of subject, as described above. The gradient and shim field maps may be directly measured for the system or accessed from previous measurement stored by a computer system.

In block 204, the target volume (e.g., 122) for imaging and target magnetization profile (e.g., 152) may be defined by a user. As a non-limiting example, the target volume may be defined as a rectangular volume in the brain region of a specific subject based on scout or localizer images acquired by or accessed by the system. The target volume may also be defined as a generic volume that corresponds to various anatomies of a wide range of subjects. For example, the target volume may be predefined for brain imaging of any given subject. The target volume may also include one or more desired slice profiles for 2D MRI. As a non-limiting example, the magnetization profile may be defined as a homogeneous flip angle within the target volume and a 0° flip angle outside the target volume. For example, the target flip angle profile may be 90° for excitation or 180° for refocusing in a spin echo or other sequence. The target flip angle may also be defined based on the Ernst angle, or other desired contrast.

The design may be constructed and performed in block 206 by a computer system. For example, an objective function can be defined by a user on a computer system based on the field map data acquired in block 202 and target volume and magnetization profile defined in block 204. As a non-limiting example, the design process may include the design 130 of FIG. 1. The design may be performed directly on the MRI console or offline on an external computer system or server. The waveforms generated (e.g., 160) can be stored by the computer system in block 208 and provided to an MRI system in block 210. Blocks 202-210 may be performed on a subject-by-subject basis or may be completed a single time for use with scanning multiple subjects. The MRI system, which may be equipped with a single transmit RF coil or several transmit RF coils configured for pTx, can use the generated waveforms to generate excitation in block 212 when a subject is present in the scanner. Generating the excitation in block 212 may include generating the freely-shaped RF pulse waveform 164 by one or more RF transmit coils and generating the gradient waveform 162 by one or more gradient coils. Generating the excitation in block 212 may further include generating a desired shim waveform 166 by driving current in one or more shim coils of the shim system. The signal elicited by the RF excitation can be acquired by the MRI system in block 214. The acquired MRI data can be processed or reconstructed to produce images, quantitative maps, or other MRI-derived data. The MRI data can be viewed on the MRI system or sent to another workstation for viewing or further processing for research or clinical purposes, and the like.

### EXAMPLES

In the following examples, examples of the method disclosed in the present disclosure are referred to as “freely-shaped pulse design”. In these examples, the freely-shaped pulse design is performed for a single transmit RF channel. Advantageously, these pulses can be played on standard systems that are not equipped with pTx systems or multichannel RF transmit coil arrays. The freely-shaped pulse design is compared with a conventional approach, an alternative advanced approach, or both. For example, the alternative approach includes a pTx pulse design in which predefined pulses are used, such as sinc or rect pulses. These pTx pulses were designed to be performed on 8-channel pTx systems.

### Example 1—Flip Angle Homogeneity

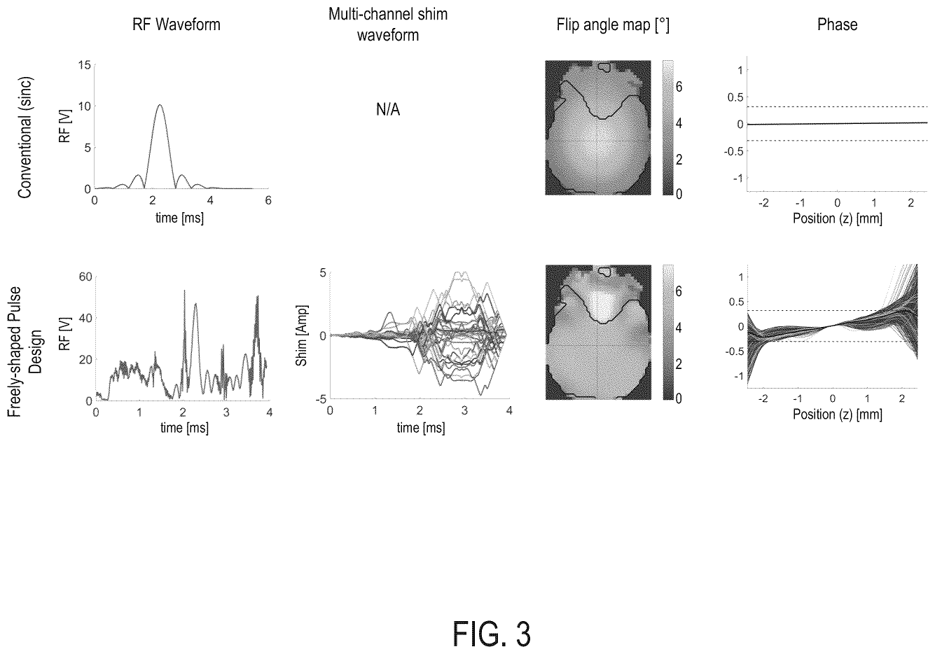

Referring now to FIG. 3, an example of slice-selective pulses is shown for use, including the RF and shim waveforms along with measured flip angle profiles achieved using the corresponding waveforms. The target excitation volume was a 5 mm 2D slice profile, and the target magnetization was a homogenous flip angle of 5° across the slice profile and a linear phase through the slice that would allow for gradient rewinding in the slice direction. The pulses were designed for and tested at 7 T.

The conventional approach (top row) utilized an RF sinc waveform and did not optimize a gradient or shim waveform. The output waveforms determined by the freely-shaped pulse design are shown in the bottom row. The pulse design included design of gradient waveforms and shim waveforms to be applied on an AC/DC multi-coil shim system. As evidenced in FIG. 3, the shapes of the RF, gradient, and shim waveforms were advantageously not constrained using the freely-shaped pulse design, and the shim waveforms were constrained for use on 50 kHz amplifiers.

As shown in FIG. 3, the use of freely-shaped pulse design reduced the flip angle inhomogeneity as compared to the conventional approach. For the slice shown, the root mean square error of the flip angle was 33% using a conventional sinc pulse and reduced to 17% using the freely-shaped pulse design disclosed herein. The improved flip angle homogeneity was achieved with a 3.9 ms RF pulse duration using the freely-shaped pulse design, which may be advantageously shorter than typical pTx pulses.

### Example 2—Slice Profile and Flip Angle Homogeneity

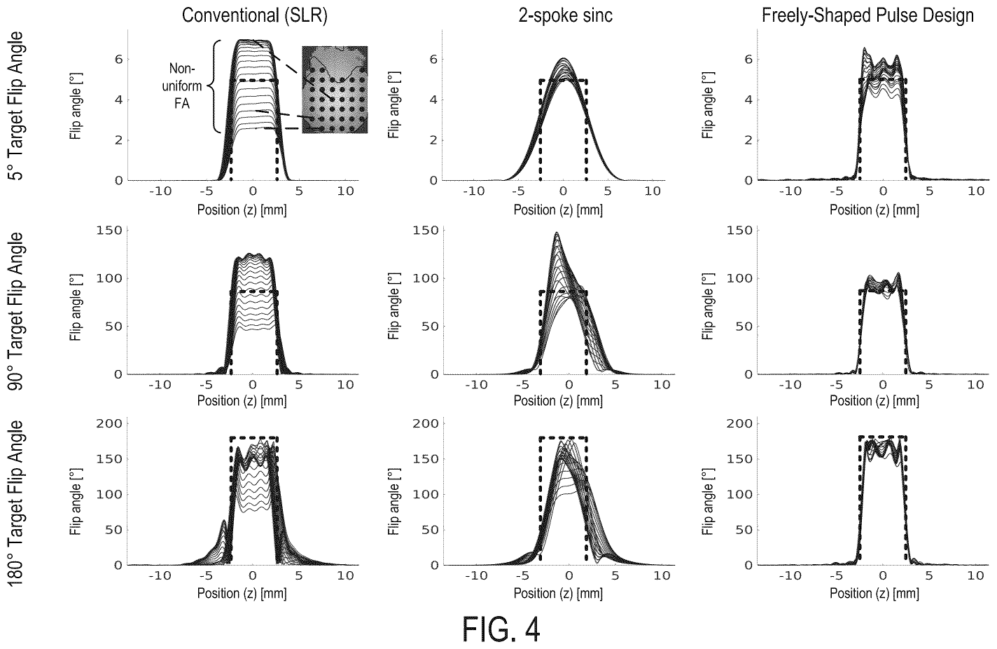

Referring now to FIG. 4, an example of slice profiles and flip angle homogeneity is shown. The slice profile is plotted for various positions within the slice. Thus, separation of the individual slice profile plots indicates non-uniform flip angles within the slice. The conventional approach (left column) used standard Shinnar-Le Roux (SLR) pulses. The 2-spoke sinc pulse approach (middle column) was performed with a single-channel RF system and included carefully-designed or even optimized shim waveforms. The freely-shaped pulse design is shown in the right column, which also included shim waveform design.

While the SLR pulses yielded reliable slice profiles at 5° and 90° flip angles, they produced severe in-slice flip angle variation at 7 T, as evidenced in FIG. 4 (right column). For larger flip angles, such as a 180° refocusing pulse, the SLR slice profile became heavily distorted due to B₁⁺ variations. The 2-spoke sinc approach achieved more uniform in-slice flip angle homogeneity but suffered from low slice selection fidelity at 5°, which may have been caused by the short duration of each spoke sub-pulse that kept the overall pulse duration below 5 ms. Moreover, the sinc pulses failed at large flip angles, such as 90° or 180°, due to the use of sinc sub-pulses, which is known to be an invalid pulse shape for slice selection of large flip-angles. Advantageously, the increased degrees of freedom of the freely-shaped pulse design yielded excellent in-slice flip angle homogeneity and slice selection fidelity.

### Example 3—Application to New Subjects

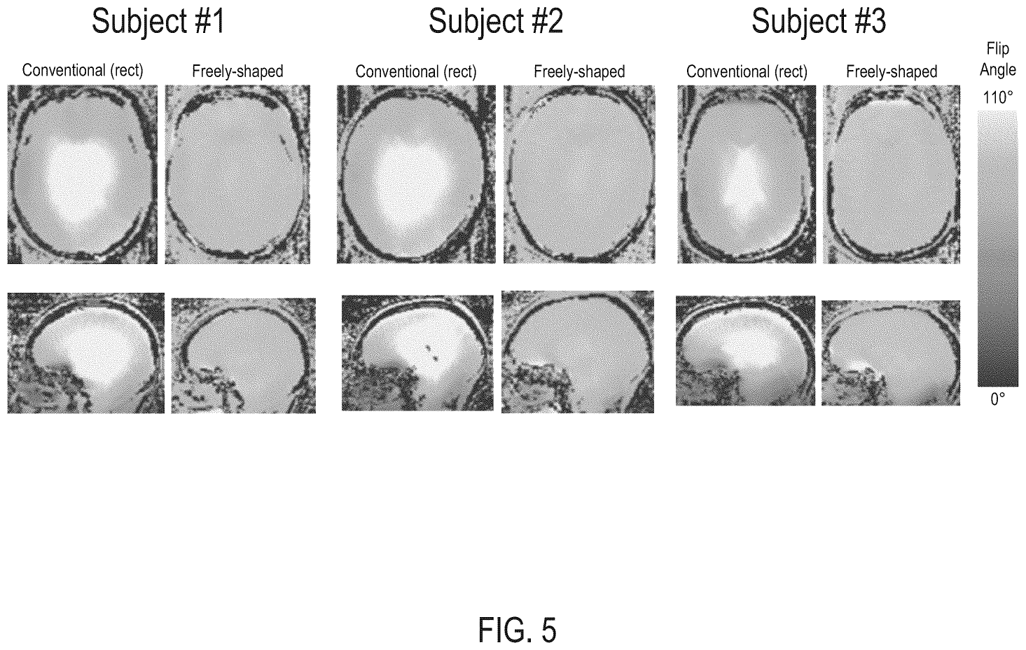

Referring now to FIG. 5, an example of flip angle profiles for transverse and sagittal slices in three different subjects imaged at 7 T is shown. The freely-shaped pulse design was performed using B₀ maps and B₁⁺ maps measured from a group of 7 subjects and applied to the three subjects shown, who were not included in the design database of field maps. The target flip angle was defined as 90°. The conventional approach shown used standard rect pulses. The conventional approach yielded an average root mean squared error of 27% compared to the 2% achieved by the freely-shaped pulse design. The results show the robust application to new subjects without requiring redesigning pulses for each unique patient.

### Example 4—Tradeoff Between SAR and Flip Angle Homogeneity

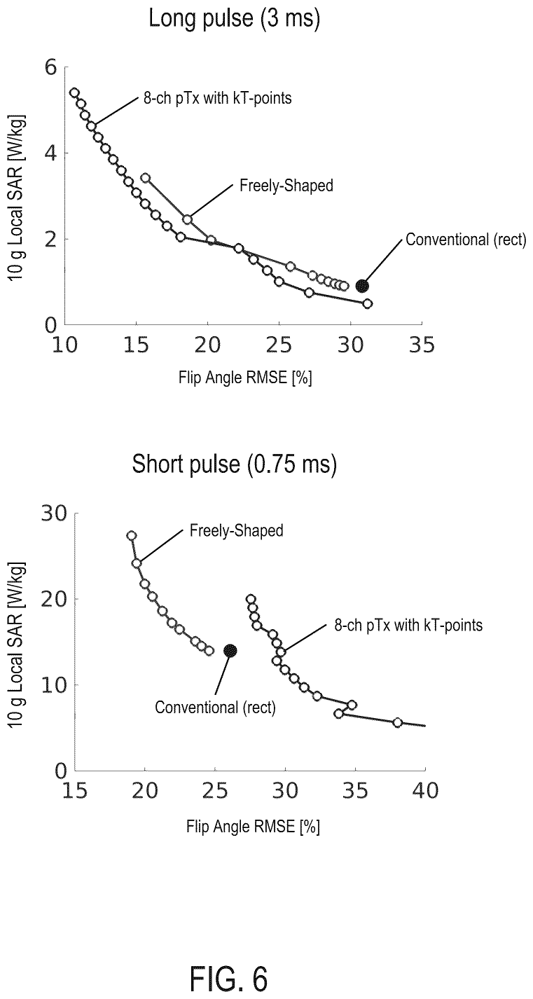

The tradeoff between SAR and flip angle RMSE is plotted in FIG. 6 as simulated for non-selective excitation at 7 T. The conventional approach used a standard rect pulse. The standard pTx approach used kT-point pulses with an 8-channel pTx system. The pTx pulse design was performed with virtual observation point SAR constraint. The freely-shaped pulse design was performed for a single channel RF transmit coil with optimization of the MRI system's linear gradient fields. The multichannel shim array was not used in this example. As FIG. 6 shows, the freely-shaped pulse design produced similar local SAR than VOP-constrained pTx kT-point pulses at a long (3 ms) pulse duration. At shorter pulse durations, such as 0.75 ms, the freely-shaped pulse design produced an advantageous SAR vs. flip angle tradeoff. Using the freely-shaped pulse design, RF and multichannel shim array current waveforms can be performed continuously without gaps, which can make the designed or even optimized pulses more RF-efficient than kT-points, which interrupt the RF pulse during the gradient blips.

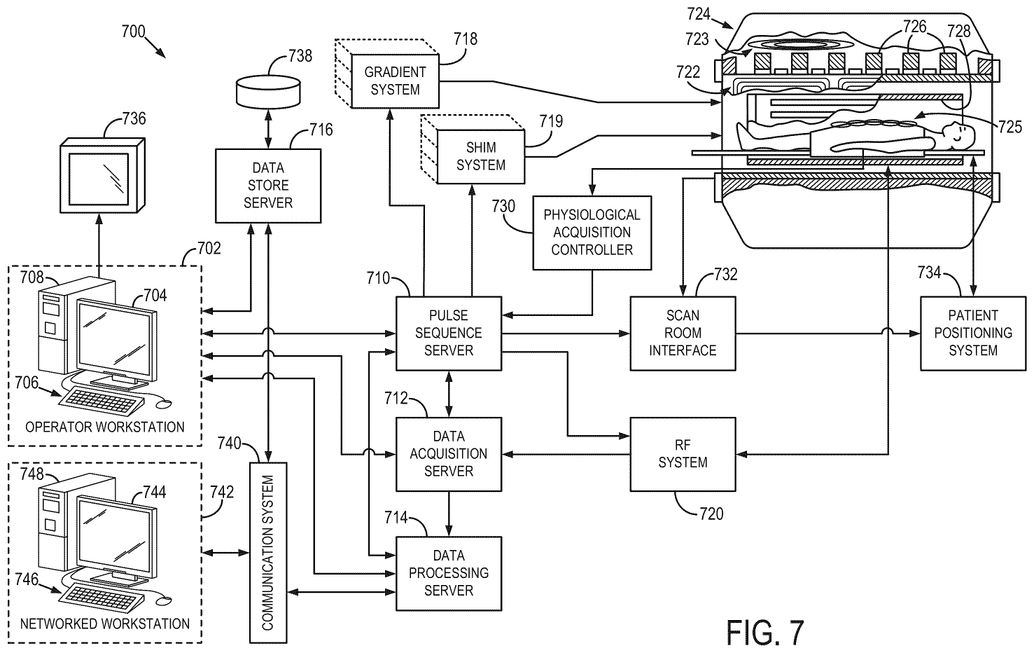

Referring particularly now to FIG. 7, an example of an MRI system 700 that can implement the methods described herein is illustrated. The MRI system 700 includes an operator workstation 702 that may include a display 704, one or more input devices 706 (e.g., a keyboard, a mouse), and a processor 708. The processor 708 may include a commercially available programmable machine running a commercially available operating system. The operator workstation 702 provides an operator interface that facilitates entering scan parameters into the MRI system 700. The operator workstation 702 may be coupled to different servers, including, for example, a pulse sequence server 710, a data acquisition server 712, a data processing server 714, and a data store server 716. The operator workstation 702 and the servers 710, 712, 714, and 716 may be connected via a communication system 740, which may include wired or wireless network connections.

The MRI system 700 also includes a magnet assembly 724 that includes a polarizing magnet 726, which may be a low-field magnet. The MRI system 700 may optionally include a whole-body RF coil 728 and a gradient system 718 that controls a gradient coil assembly 722. The MRI system 700 may also include a shim system 719 that controls a shim coil assembly 723 and/or 725.

The pulse sequence server 710 functions in response to instructions provided by the operator workstation 702 to operate a gradient system 718, a shim system 719, and a radiofrequency (“RF”) system 720. Gradient waveforms for performing a prescribed scan are produced and applied to the gradient system 718, which then excited gradient coils in an assembly 722 to produce the magnetic field gradients (e.g., Gₓ, G_{y}, and G_{z}) that can be used for spatially encoding magnetic resonance signals. Shim waveforms for shaping the magnetic field are produced and applied to the shim system 719, which then drive the shim coil assembly 723 and/or 725. The gradient coil assembly 722 and shim coil assembly 723 may form part of a magnet assembly 724 that includes a polarizing magnet 726 and a whole-body RF coil 728. The shim coil assembly 725 may also include one or more external shim coils arranged in a shim coil array.

RF waveforms are applied by the RF system 720 to the RF coil 728, or a separate local coil to perform the prescribed magnetic resonance pulse sequence. Responsive magnetic resonance signals detected by the RF coil 728, or a separate local coil, are received by the RF system 720. The responsive magnetic resonance signals may be amplified, demodulated, filtered, and digitized under direction of commands produced by the pulse sequence server 710. The RF system 720 includes an RF transmitter for producing a wide variety of RF pulses used in MRI pulse sequences. The RF transmitter is responsive to the prescribed scan and direction from the pulse sequence server 710 to produce RF pulses of the desired frequency, phase, and pulse amplitude waveform. The generated RF pulses may be applied to the whole-body RF coil 728 or to one or more local coils or coil arrays.

The RF system 720 also includes one or more RF receiver channels. An RF receiver channel includes an RF preamplifier that amplifies the magnetic resonance signal received by the coil 728 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M=\sqrt{(I²+Q²)}

and the phase of the received magnetic resonance signal may also be determined according to the following relationship:

$$ϕ = {tan}^{- 1} ( \frac{Q}{I} )$$

The pulse sequence server 710 may receive patient data from a physiological acquisition controller 730. By way of example, the physiological acquisition controller 730 may receive signals from a number of different sensors connected to the patient, including electrocardiograph (“ECG”) signals from electrodes, or respiratory signals from a respiratory bellows or other respiratory monitoring devices. These signals may be used by the pulse sequence server 710 to synchronize, or “gate,” the performance of the scan with the subject's heartbeat or respiration.

The pulse sequence server 710 may also connect to a scan room interface circuit 732 that receives signals from various sensors associated with the condition of the patient and the magnet system. Through the scan room interface circuit 732, a patient positioning system 734 can receive commands to move the patient to desired positions during the scan.

The digitized magnetic resonance signal samples produced by the RF system 720 are received by the data acquisition server 712. The data acquisition server 712 operates in response to instructions downloaded from the operator workstation 702 to receive the real-time magnetic resonance data and provide buffer storage, so that data are not lost by data overrun. In some scans, the data acquisition server 712 passes the acquired magnetic resonance data to the data processor server 714. In scans that require information derived from acquired magnetic resonance data to control the further performance of the scan, the data acquisition server 712 may be programmed to produce such information and convey it to the pulse sequence server 710. For example, during pre-scans, magnetic resonance data may be acquired and used to calibrate the pulse sequence performed by the pulse sequence server 710. As another example, navigator signals may be acquired and used to adjust the operating parameters of the RF system 720 or the gradient system 718, or to control the view order in which k-space is sampled. In still another example, the data acquisition server 712 may also process magnetic resonance signals used to detect the arrival of a contrast agent in a magnetic resonance angiography (“MRA”) scan. For example, the data acquisition server 712 may acquire magnetic resonance data and processes it in real-time to produce information that is used to control the scan.

The data processing server 714 receives magnetic resonance data from the data acquisition server 712 and processes the magnetic resonance data in accordance with instructions provided by the operator workstation 702. Such processing may include, for example, reconstructing two-dimensional or three-dimensional images by performing a Fourier transformation of raw k-space data, performing other image reconstruction algorithms (e.g., iterative or back projection reconstruction algorithms), applying filters to raw k-space data or to reconstructed images, generating functional magnetic resonance images, or calculating motion or flow images.

Images reconstructed by the data processing server 714 are conveyed back to the operator workstation 702 for storage. Real-time images may be stored in a data base memory cache, from which they may be output to operator display 702 or a display 736. Batch mode images or selected real time images may be stored in a host database on disc storage 738. When such images have been reconstructed and transferred to storage, the data processing server 714 may notify the data store server 716 on the operator workstation 702. The operator workstation 702 may be used by an operator to archive the images, produce films, or send the images via a network to other facilities.

The MRI system 700 may also include one or more networked workstations 742. For example, a networked workstation 742 may include a display 744, one or more input devices 746 (e.g., a keyboard, a mouse), and a processor 748. The networked workstation 742 may be located within the same facility as the operator workstation 702, or in a different facility, such as a different healthcare institution or clinic.

The networked workstation 742 may gain remote access to the data processing server 714 or data store server 716 via the communication system 740. Accordingly, multiple networked workstations 742 may have access to the data processing server 714 and the data store server 716. In this manner, magnetic resonance data, reconstructed images, or other data may be exchanged between the data processing server 714 or the data store server 716 and the networked workstations 742, such that the data or images may be remotely processed by a networked workstation 742.

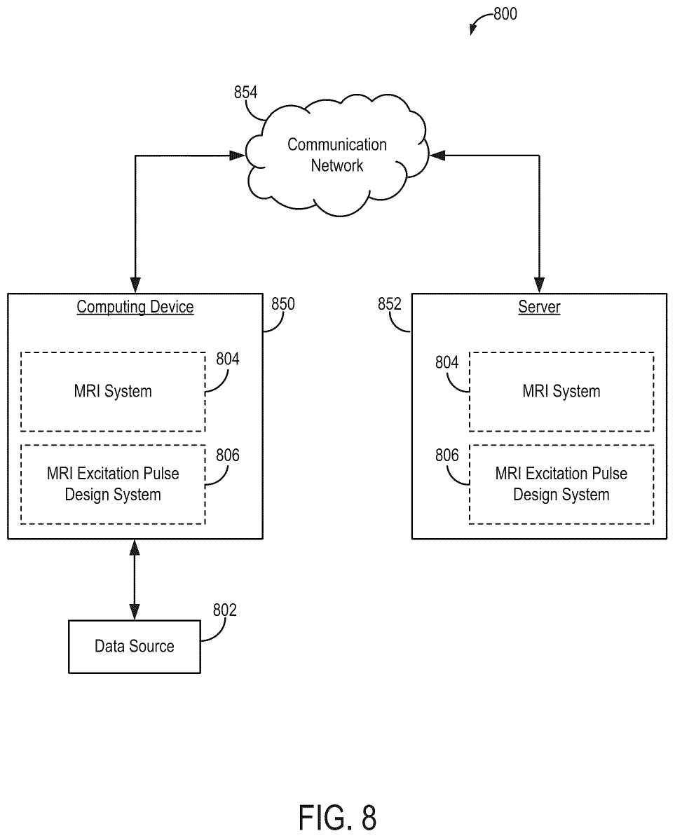

Referring now to FIG. 8, an example of an MRI system 800 is shown, which may be used in accordance with some aspects of the systems and methods described in the present disclosure. As shown in FIG. 8, a computing device 850 can receive one or more types of data (e.g., signal evolution data, k-space data, receiver coil sensitivity data) from data source 802. In some configurations, computing device 850 can execute at least a portion of an MRI system 804 to reconstruct images from magnetic resonance data (e.g., k-space data) acquired using the freely-shaped excitation pulses. The computing device 850 can also execute at least a portion of an MRI excitation pulse design system 806 to determine the freely-shaped pulses for the MRI system 804. In some configurations, the MRI excitation pulse design system 804 can implement an automated pipeline to provide MRI images, quantitative maps, etc.

Additionally or alternatively, in some configurations, the computing device 850 can communicate information about data received from the data source 802 to a server 852 over a communication network 854, which can execute at least a portion of the MRI system 804 and the MRI excitation pulse design system 806. In such configurations, the server 852 can return information to the computing device 850 (and/or any other suitable computing device) indicative of an output of the MRI system 804 or the MRI excitation pulse design system 806.

In some configurations, computing device 850 and/or server 852 can be any suitable computing device or combination of devices, such as a desktop computer, a laptop computer, a smartphone, a tablet computer, a wearable computer, a server computer, a virtual machine being executed by a physical computing device, and so on. The computing device 850 and/or server 852 can also reconstruct images from the data.

In some configurations, data source 802 can be any suitable source of data (e.g., measurement data, images reconstructed from measurement data, processed image data), such as an MRI system, another computing device (e.g., a server storing measurement data, images reconstructed from measurement data, processed image data), and so on. In some configurations, data source 802 can be local to computing device 850. For example, data source 802 can be incorporated with computing device 850 (e.g., computing device 850 can be configured as part of a device for measuring, recording, estimating, acquiring, or otherwise collecting or storing data). As another example, data source 802 can be connected to computing device 850 by a cable, a direct wireless link, and so on. Additionally or alternatively, in some configurations, data source 802 can be located locally and/or remotely from computing device 850, and can communicate data to computing device 850 (and/or server 852) via a communication network (e.g., communication network 854).

In some configurations, communication network 854 can be any suitable communication network or combination of communication networks. For example, communication network 854 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some configurations, communication network 854 can be a local area network, a wide area network, a public network (e.g., the Internet), a private or semi-private network (e.g., a corporate or university intranet), any other suitable type of network, or any suitable combination of networks. Communications links shown in FIG. 8 can each be any suitable communications link or combination of communications links, such as wired links, fiber optic links, Wi-Fi links, Bluetooth links, cellular links, and so on.

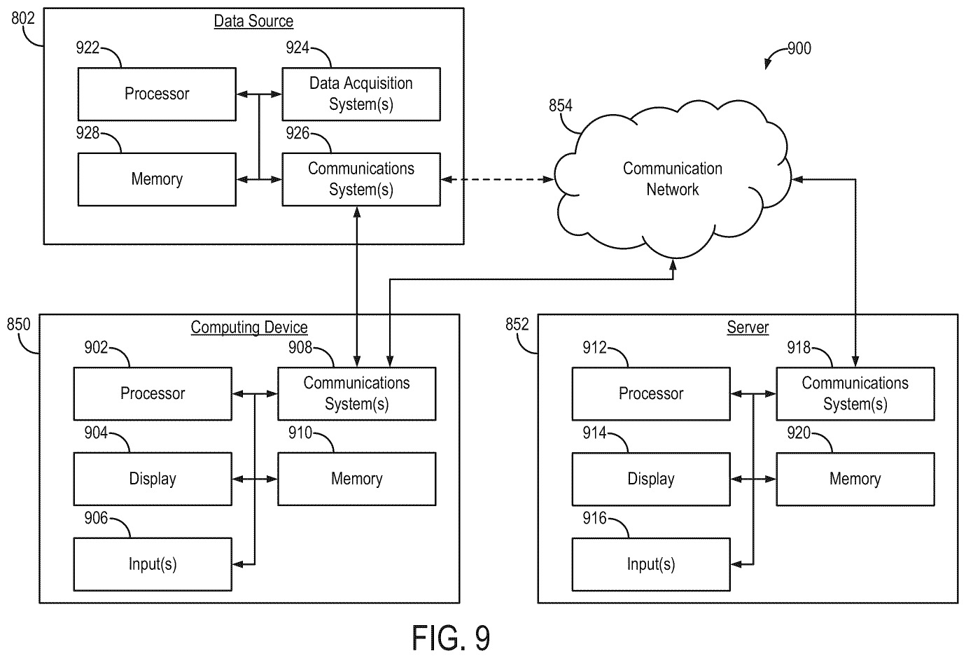

Referring now to FIG. 9, an example of hardware 900 that can be used to implement data source 802, computing device 850, and server 852 in accordance with some configurations of the systems and methods described in the present disclosure is shown.

As shown in FIG. 9, in some configurations, computing device 850 can include a processor 902, a display 904, one or more inputs 906, one or more communication systems 908, and/or memory 910. In some configurations, processor 902 can be any suitable hardware processor or combination of processors, such as a central processing unit (“CPU”), a graphics processing unit (“GPU”), and so on. In some configurations, display 904 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some configurations, inputs 906 can include any suitable input devices and/or sensors that can be used to receive user input, such as a keyboard, a mouse, a touchscreen, a microphone, and so on.

In some configurations, communications systems 908 can include any suitable hardware, firmware, and/or software for communicating information over communication network 854 and/or any other suitable communication networks. For example, communications systems 908 can include one or more transceivers, one or more communication chips and/or chip sets, and so on. In a more particular example, communications systems 908 can include hardware, firmware, and/or software that can be used to establish a Wi-Fi connection, a Bluetooth connection, a cellular connection, an Ethernet connection, and so on.

In some configurations, memory 910 can include any suitable storage device or devices that can be used to store instructions, values, data, or the like, that can be used, for example, by processor 902 to present content using display 904, to communicate with server 852 via communications system(s) 908, and so on. Memory 910 can include any suitable volatile memory, non-volatile memory, storage, or any suitable combination thereof. For example, memory 910 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some configurations, memory 910 can have encoded thereon, or otherwise stored therein, a computer program for controlling operation of computing device 850. In such configurations, processor 902 can execute at least a portion of the computer program to present content (e.g., images, user interfaces, graphics, tables), receive content from server 852, transmit information to server 852, and so on. For example, the processor 902 and the memory 910 can be configured to perform the methods described herein.

In some configurations, server 852 can include a processor 912, a display 914, one or more inputs 916, one or more communications systems 918, and/or memory 920. In some configurations, processor 912 can be any suitable hardware processor or combination of processors, such as a CPU, a GPU, and so on. In some configurations, display 914 can include any suitable display devices, such as an LCD screen, LED display, OLED display, electrophoretic display, a computer monitor, a touchscreen, a television, and so on. In some configurations, inputs 916 can include any suitable input devices and/or sensors that can be used to receive user input, such as a keyboard, a mouse, a touchscreen, a microphone, and so on.

In some configurations, communications systems 918 can include any suitable hardware, firmware, and/or software for communicating information over communication network 854 and/or any other suitable communication networks. For example, communications systems 918 can include one or more transceivers, one or more communication chips and/or chip sets, and so on. In a more particular example, communications systems 918 can include hardware, firmware, and/or software that can be used to establish a Wi-Fi connection, a Bluetooth connection, a cellular connection, an Ethernet connection, and so on.

In some configurations, memory 920 can include any suitable storage device or devices that can be used to store instructions, values, data, or the like, that can be used, for example, by processor 912 to present content using display 914, to communicate with one or more computing devices 850, and so on. Memory 920 can include any suitable volatile memory, non-volatile memory, storage, or any suitable combination thereof. For example, memory 920 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 920 can have encoded thereon a server program for controlling operation of server 852. In such configurations, processor 912 can execute at least a portion of the server program to transmit information and/or content (e.g., data, images, a user interface) to one or more computing devices 850, receive information and/or content from one or more computing devices 850, receive instructions from one or more devices (e.g., a personal computer, a laptop computer, a tablet computer, a smartphone), and so on.

In some configurations, the server 852 is configured to perform the methods described in the present disclosure. For example, the processor 912 and memory 920 can be configured to perform the methods described herein.

In some configurations, data source 802 can include a processor 922, one or more data acquisition systems 924, one or more communications systems 926, and/or memory 928. In some configurations, processor 922 can be any suitable hardware processor or combination of processors, such as a CPU, a GPU, and so on. In some configurations, the one or more data acquisition systems 924 are generally configured to acquire data, images, or both, and can include an MRI system. Additionally or alternatively, in some configurations, the one or more data acquisition systems 924 can include any suitable hardware, firmware, and/or software for coupling to and/or controlling operations of an MRI system. In some configurations, one or more portions of the data acquisition system(s) 924 can be removable and/or replaceable.

Note that, although not shown, data source 802 can include any suitable inputs and/or outputs. For example, data source 802 can include input devices and/or sensors that can be used to receive user input, such as a keyboard, a mouse, a touchscreen, a microphone, a trackpad, a trackball, and so on. As another example, data source 802 can include any suitable display devices, such as an LCD screen, an LED display, an OLED display, an electrophoretic display, a computer monitor, a touchscreen, a television, etc., one or more speakers, and so on.

In some configurations, communications systems 926 can include any suitable hardware, firmware, and/or software for communicating information to computing device 850 (and, in some configurations, over communication network 854 and/or any other suitable communication networks). For example, communications systems 926 can include one or more transceivers, one or more communication chips and/or chip sets, and so on. In a more particular example, communications systems 926 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some configurations, memory 928 can include any suitable storage device or devices that can be used to store instructions, values, data, or the like, that can be used, for example, by processor 922 to control the one or more data acquisition systems 924, and/or receive data from the one or more data acquisition systems 924; to generate images from data; present content (e.g., data, images, a user interface) using a display; communicate with one or more computing devices 850; and so on. Memory 928 can include any suitable volatile memory, non-volatile memory, storage, or any suitable combination thereof. For example, memory 928 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 928 can have encoded thereon, or otherwise stored therein, a program for controlling operation of medical image data source 802. In such configurations, processor 922 can execute at least a portion of the program to generate images, transmit information and/or content (e.g., data, images, a user interface) to one or more computing devices 850, receive information and/or content from one or more computing devices 850, receive instructions from one or more devices (e.g., a personal computer, a laptop computer, a tablet computer, a smartphone, etc.), and so on.

In some configurations, any suitable computer-readable media can be used for storing instructions for performing the functions and/or processes described herein. For example, in some configurations, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

1. A method for performing a magnetic resonance imaging (MRI) process, the method comprising:
(a) determining a target excitation region for an imaging process of a subject located within the MRI system;
(b) determining a target magnetization profile for the imaging process;
(c) accessing field map data with a computer system, wherein the field map data indicate a B₀ field map measured by the MRI system and a B₁⁺ field map measured for a radiofrequency (RF) transmit coil of the MRI system;
(d) with the computer system, designing an RF pulse waveform with shape selected using an objective function having at least one constraint, and wherein the objective function is configured to control at least one of a deviation from the target magnetization profile within the target excitation region or an RF power requirement level based on the field map data;
(e) communicating the RF pulse waveform for use with the MRI system to perform an imaging process of the subject.

2. The method as recited in claim 1, wherein the RF pulse waveform is free of sinc and rect pulses.

3. The method as recited in claim 1, wherein the MRI system comprises a single RF transmit coil configured to generate the RF excitation pulse.

4. The method as recited in claim 1, wherein the MRI system comprises two or more RF transmit coils configured for parallel transmit.

5. The method as recited in claim 1, further comprising applying the RF pulse waveform by an RF system of the MRI system, applying a gradient waveform by a gradient system of the MRI system, and acquiring MRI data from a subject to perform the imaging process.

6. The method as recited in claim 1, wherein the at least one constraint comprises at least one of a slew rate of a gradient waveform or an acceleration of the gradient waveform.

7. The method as recited in claim 1, wherein the target excitation region is a slice profile, and wherein the MRI system is configured for 2D imaging.

8. The method as recited in claim 1, wherein the target excitation region is a 3D volume of interest, and wherein the MRI system is configured for 3D imaging.

9. The method as recited in claim 1, wherein the target magnetization profile is defined as a homogeneous flip angle within the target excitation volume.

10. The method as recited in claim 9, wherein the deviation from the target magnetization profile represents Be inhomogeneity.

11. The method as recited in claim 1, wherein the target magnetization profile is defined inside the target excitation region and outside the target excitation region and designing the RF pulse waveform further includes control a deviation from the target magnetization profile outside the target excitation region.

12. The method as recited in claim 11, wherein the target magnetization profile is defined as a 0° flip angle outside the target excitation region.

13. The method as recited in claim 1, further comprising generating at least one of a gradient waveform or a shim waveform, and wherein the objective function is further constrained by at least one of a slew rate of a gradient waveform, an acceleration of the gradient waveform, a slew rate of the shim waveform, or an acceleration of the shim waveform.

14. The method as recited in claim 1, wherein the B₀ field map comprises a B₀ field map measured for a single subject and the B₁⁺ field map comprises a B₁⁺ field map measured for the single subject.

15. The method as recited in claim 1, wherein the B₀ field map comprises a plurality of B₀ field maps measured for a plurality of subjects and the B₁⁺ field map comprises a plurality of B₁⁺ field maps measured for a plurality of subjects.

16. The method as recited in claim 1, wherein the objective function is further configured to control a specific absorption rate (SAR).

17. A magnetic resonance imaging (MRI) system, the system comprising:
a magnetic system configured to produce a main magnetic (B₀) field across at least a portion of a subject to be imaged with the MRI system;
a radiofrequency (RF) system configured to transmit and receive a radiofrequency (B₁⁺) field across at least a target region in the portion of the subject;
a gradient system configured to spatially encode the B₁⁺ field using a gradient waveform; and
a control system configured to control the RF system to generate an RF excitation pulse using the RF system and having a shape selected by penalizing deviation of a flip-angle of the RF excitation pulse from a target flip-angle distribution to achieve a target magnetization profile in the target region.

18. The MRI system as recited in claim 17, wherein the shape of the excitation pulse is constrained by the gradient waveform.

19. The MRI system as recited in claim 18, wherein constraining the shape of the excitation pulse by the gradient waveform includes constraining the shape of the excitation pulse based on at least one of a slew rate or an acceleration of the gradient waveform.

20. The MRI system as recited in claim 17, wherein the RF excitation pulse and the gradient waveform are configured to control at least one of a specific absorption rate (SAR) or a deviation from the target magnetization profile over a target region in the portion of the subject.

21. The MRI system as recited in claim 17, wherein the RF excitation pulse is free of sinc and rect pulses.

22. The MRI system as recited in claim 17, wherein the RF system comprises a single RF transmit coil configured to generate the RF excitation pulse.

23. The MRI system as recited in claim 17, wherein the RF system comprises two or more RF transmit coils configured to generate the RF excitation pulse using parallel transmit.

24. The MRI system as recited in claim 17, wherein the target magnetization profile is defined inside and outside of the target volume and the optimization is further configured to control a deviation from the target magnetization profile outside of the target volume.

25. The MRI system as recited in claim 17, wherein the optimization is constrained by at least one of a slew rate of the gradient waveform or an acceleration of the gradient waveform.

26. The MRI system as recited in claim 17, wherein the target volume is a slice profile, and wherein the MRI system is configured for 2D MRI.

27. The MRI system as recited in claim 17, wherein the target volume is a volume of interest, and wherein the MRI system is configured for 3D MRI.

28. The MRI system as recited in claim 17, wherein the system further comprises a shim system configured to shape the B₀ field, and wherein the control system is further configured to control the shim system to generate a shim waveform to reduce at least one of a power consumption or a deviation from the target magnetization profile.

29. The MRI system as recited in claim 17, wherein the control system is further configured to perform an optimization using a B₀ field map that comprises a B₀ field map measured for the subject and a B₁⁺ field map that comprises a B₁⁺ field map measured for the subject to design the RF pulse.

30. The MRI system as recited in claim 17, wherein the control system is further configured to perform an optimization using a B₀ field map that comprises a plurality of B₀ field maps measured for a plurality of subjects and a B₁⁺ field map that comprises a plurality of B₁⁺ field maps measured for a plurality of subjects.
